(19) United States
(12) Patent Application Publication
(10) Pub. No.: US 2011/0081410 A1
(43) Pub. Date: Apr. 7, 2011

(54) THERAPEUTIC AGENT FOR LOCAL INFLAMMATION

(75) Inventor: Rie Igarashi, Nara-shi (JP)

(73) Assignee: ST. MARIANNA UNIVERSITY, SCHOOL OFMEDICINE, Kawasaki-shi (JP)

(21) Appl. No.: 11/993,209

(22) PCT Filed: Jun. 23, 2006
(86) PCT No.: PCT/JP2006/312570

§ 371 Date: Nov. 12, 2010

(30) Foreign Application Priority Data
Jun. 28, 2005 (JP) 2005-188605

(51) Int. Cl. A61K 9/48; A61K 31/203; A61K 31/07; A61P 29/00; A61P 19/02

(52) U.S. Cl. 424/451; 514/559; 514/725

(57) ABSTRACT

The agents of the present invention comprise, as a main ingredient, a polyvalent metal inorganic-salt nanocapsule which encapsulates a retinoid such as retinoic acid. The agents could penetrate into a joint when applied to the skin and induce hyaluronic acid production in a synovial membrane or chondrocyte. Moreover, application of the nanocapsule of the present invention to the skin for a certain period of time lowered the values of inflammatory cytokines and MMPs in the blood.

### TECHNICAL FIELD

The present invention relates to therapeutic agents for local inflammation, particularly therapeutic agents for local inflammation comprising a retinoid-containing capsule as a main ingredient.

### BACKGROUND ART

Osteoarthritis (OA) is a disease where articular cartilages are deformed mostly by aging, causing pains and movement disorders. Normal articular cartilages are kept elastic by repetitive metabolism, but become less elastic with age and are gradually worn out. As a result, bones which constitute a joint are in direct contact with each other; ligaments and joint capsules are loosened, tensioned, and/or compressed; and these cause pain. As it progresses, eventually, rubbing of bones generates spinal spurs on bones, causing more pain. The main cause of OA is aging. Now that a full-fledged aging society has come, a rapid increase in the number of OA patients is predicted.

For OA treatment, physical therapies and drug therapies by drug administration/injection are employed. If there is no symptomatic improvement, operations are performed. One of the drug therapies for OA is a direct injection of hyaluronic acid, a protective agent for articular cartilage, into the affected part. The intra-articular injection of hyaluronic acid can prevent rubbing of bones to remove pain. The improvement of joints by hyaluronic acid injection is an excellent therapeutic method which can restore the patient's QOL. However, in order to attain the therapeutic effect of this method, the hyaluronic acid injection is normally required once a week for about five weeks. Moreover, the attained analgesic effect is not permanent, and treatment needs to be recommenced after a certain period of time, which increases the therapeutic burden on the patient.

Meanwhile, hyaluronic acid is an ingredient that makes the skin elastic, and is also known to have an effect of improving skin aging symptoms such as freckles and wrinkles. Thus, hyaluronic acid is blended and used in cosmetics and the like. It is also revealed that hyaluronic acid production is induced by retinoic acid in vivo (Patent Document 1). In recent years, for the purpose of improving freckles and wrinkles, hyaluronic acid is induced in vivo by retinoic acid, rather than being directly used. The present inventors have so far developed retinoic acid-containing nanocapsules, and elucidated that applying them to the skin (upper stratum corneum), which allows retinoic acid to penetrate the stratum corneum and reach epidermal cells to thereby induce hyaluronic acid production in the epidermal cells, can improve freckles and wrinkles (Patent Document 2 and Non Patent Document 1). Moreover, control of the particle size of the nanocapsule mentioned above was successfully achieved by improving the production method of the nanocapsule (Patent Document 3).

[Patent Document 1] Japanese Patent Kohyo Publication No. (JP-A) H09-503499 (unexamined Japanese national phase publication corresponding to a non-Japanese international publication)

[Patent Document 2] Japanese Patent Application Kokai Publication No. (JP-A) 2004-161739 (unexamined, published Japanese patent application)

[Patent Document 3] WO2005/037268

[Non Patent Document 1] Journal of Controlled Release, vol. 104-1, pp 29-40 (2005)

### DISCLOSURE OF THE INVENTION

### Problems to be Solved by the Invention

An objective of the present invention is to provide novel external preparations which can alleviate periarticular local inflammation such as osteoarthritis.

### Means for Solving the Problems

To address the above problem, the present inventors have dedicated themselves to research whether the developed retinoic acid nanocapsule is applicable to the treatment of arthritis. As a result, it was found that in in vitro experiments, the retinoic acid nanocapsule is able to produce hyaluronic acid in a synovial cell using synovial cells from OA patients. Furthermore, in experiments using arthritis model animals, application of the retinoic acid nanocapsule to the skin surface of a joint was able to induce hyaluronic acid in a synovial membrane/chondrocyte within the joint, and improve arthritis. Although for conventional retinoic acid, there has been no report on notable effects of its percutaneous application, percutaneous application of the retinoic acid nanocapsule of the present invention to arthritis model animals showed remarkable effects. No effect was observed in the intravenous injection of the retinoic acid nanocapsule performed at the same time, while local administration of the nanocapsule of the present invention was shown to exert a particularly excellent effect. As a further surprising result, it was elucidated that the retinoic acid nanocapsule itself has an ability to inhibit the production of inflammatory cytokines, TNF-α, IL-6, and IL-1α, and furthermore inhibits the production of MMP-3 which has a degrading effect on articular cartilage and proteoglycan.

These findings showed that the retinoic acid nanocapsule is effective as an agent that inhibits the accompanying inflammation of destruction of cartilage tissue, such as rheumatoid arthritis (RA), and useful for the treatment of osteoarthritis. The present invention is based on these novel findings, and specifically shown below.

[1] A therapeutic agent for local inflammation comprising an inorganic salt capsule which encapsulates a retinoid;

[2] The therapeutic agent for local inflammation according to [1], wherein the inorganic salt capsule has a hydrophilic group and a polyvalent metal salt on its surface;

[3] The therapeutic agent for local inflammation according to [1], wherein the retinoid is a retinoic acid, a retinol, or a retinol derivative;

[4] The therapeutic agent for local inflammation according to [1], wherein the inorganic salt capsule is nano size;

[5] The therapeutic agent for local inflammation according to [2], wherein the hydrophilic group is a polyoxyethylene group or a sugar chain;

[6] The therapeutic agent for local inflammation according to [2], wherein the polyvalent metal is a divalent or trivalent metal;

[7] The therapeutic agent for local inflammation according to [2], wherein the polyvalent metal is calcium, magnesium, zinc, aluminum, or copper;

[8] The therapeutic agent for local inflammation according to [2], wherein the polyvalent metal salt is a carbonate, phosphate, or sulfate of the polyvalent metal;

[9] The therapeutic agent for local inflammation according to [1], wherein the local inflammation is associated with the production of MMP and/or an inflammatory cytokine;

[10] The therapeutic agent for local inflammation according to [9], wherein the MMP is MMP-3;

[11] The therapeutic agent for local inflammation according to [9], wherein the inflammatory cytokine is TNF-α, IL-6, and/or IL-1α;

[12] The therapeutic agent for local inflammation according to [1], wherein the local inflammation is arthritis;

[13] The therapeutic agent for local inflammation according to [1], the agent is an external preparation;

[14] A preventive and/or therapeutic agent for a joint disease that accompanies a decrease in intra-articular hyaluronic acid level, comprising as an active ingredient, a nanocapsule comprising a micelle particle that is formed by one or more retinoid-containing amphiphilic substances and coated with a polyvalent metal inorganic salt, wherein a hydrophilic group from at least one of the amphiphilic substances forming said micelle is at least partially present on the polyvalent metal inorganic salt-coated surface;

[15] The preventive and/or therapeutic agent of [14], wherein one or more amphiphilic substances contains at least a retinoid and one or more nonionic surfactants;

[16] The preventive and/or therapeutic agent of [15], wherein the nonionic surfactant is a polyoxyethylene-based nonionic surfactant or a sucrose fatty acid ester;

[17] The preventive and/or therapeutic agent of [15], wherein the hydrophilic group of the nonionic surfactant is a polyoxyethylene chain;

[18] The preventive and/or therapeutic agent of any one of [14] to [17], wherein the joint disease that accompanies a decrease in intra-articular hyaluronic acid level is knee joint pain in osteoarthritis, scapulohumeral periarthritis, or chronic rheumatoid arthritis;

[19] A method for preventing and/or or treating a joint disease or local inflammation that accompanies a decrease in hyaluronic acid level, comprising administration of the agent of any one of [1] to [18]; and

[20] Use of a nanocapsule comprising a spherical micelle that is formed by one or more retinoid-containing amphiphilic substances and coated with a polyvalent metal inorganic salt, wherein a hydrophilic group from at least one of the amphiphilic substances forming said micelle projects from the polyvalent metal salt-coated surface, for the production of a preventive and/or therapeutic agent for a joint disease or local inflammation that accompanies a decrease in hyaluronic acid level.

### BRIEF DESCRIPTION OF THE DRAWINGS

FIG. 1 presents photographs of in vitro cell culture experiments showing analysis results on the activity of the retinoic acid nanocapsule to induce hyaluronic acid production.

FIG. 2 presents photographs of in vivo experiments showing conditions of affected parts after applying external preparations of retinoic acid nanocapsule and control to the front paw joint and hind paw joint of arthritis-induced mice for a fixed period.

FIG. 3 presents graphs showing measured values of IL-6 and MMP-3 in the blood, and measured values of the percentage of collagen content in the affected parts after applying external preparations of retinoic acid nanocapsule and control to the front paw joint and hind paw joint of arthritis-induced mice.

FIG. 4 presents photographs showing results of colloidal iron staining of the histological sections of joints collected after external preparations of retinoic acid nanocapsule and control were applied to the front paw joint and hind paw joint of arthritis-induced mice for a fixed period.

FIG. 5 presents graphs showing measured values of TNF-α (a) and IL-1α (b) in the blood after external preparations of retinoic acid nanocapsule and control were applied to the front paw joint and hind paw joint of arthritis-induced mice.

### BEST MODE FOR CARRYING OUT THE INVENTION

The present invention is described more in detail with reference to suitable Examples.

In the present invention, therapeutic agents for local inflammation comprise as a main ingredient an inorganic-salt nanocapsule that encapsulates a retinoid. That is, the medicinal ingredient in the agents of the present invention is a retinoid. Generally, retinoid is a generic name for compounds with a vitamin A (retinol) skeleton. It is also a generic name for compounds having the same or similar effects as retinoic acid (all-trans and 9-cis), which is an active form of retinol and binds to a retinoic acid receptor. In this case, the chemical structure may largely differ from that of retinoic acid in terms of the appearance (“Seikagaku Jiten (Dictionary of Biochemistry)” third edition, published by Tokyo Kagaku Dojin)). The retinoid in the present invention includes retinoic acids, retinols, and derivatives thereof. Of them, retinoic acid is a suitable example of the retinoid of the present invention because of its high activity to induce hyaluronic acid production in a synovial membrane or chondrocyte. The retinol derivative mentioned above suitably includes retinol palmitate, and may also include retinol acetate and the like, so long as it has the activity to induce hyaluronic acid production. The retinoic acid and retinol are known to be in all-trans form, cis form, and the like, but the all-trans form is suitable. The cis form includes 13-cis, 11-cis, 9-cis, 9,13-dicis, and 11,13-dicis forms, and these various cis forms are also usable as the retinoid of the present invention so long as they are capable of inducing hyaluronic acid production in a synovial membrane or chondrocyte.

Whether or not these retinoids are capable of inducing hyaluronic acid in a synovial membrane/chondrocyte can be confirmed with reference to Example 2 described later. Specifically, a retinoid whose activity is to be confirmed is added to a culture solution of synovial cells, and the cells are cultured. After a predetermined time period, colloidal staining can be performed to confirm the activity of hyaluronic acid induction, based on the amount of black-stained particles.

The retinoid in the present invention is preferably amphiphilic. An amphiphilic retinoid is capable of forming a micelle in solution, and thus is preferred for the preparation of inorganic-salt nanocapsules.

The retinoid in the present invention preferably has properties such as inhibitory effect on production of inflammatory cytokines, and inhibitory effect on production of matrix metalloproteases (MMPs) which have a degrading effect on articular cartilage and proteoglycan.

Inflammatory cytokines produced at the time of periarticular inflammations such as osteoarthritis and rheumatoid arthritis include, for example, TNF-α, IL-1α, and IL-6. A retinoid that can at least inhibit cytokines produced at the time of inflammation is preferably used as an active ingredient of the present invention. The use of such retinoid can provide an alleviating effect on inflammation, and improve joints by inducing hyaluronic acid production as mentioned above. There are matrix metalloproteases MMPs-1 to 9, but a retinoid capable of inhibiting the production of at least MMP-3, which has effects of destructing chondrocytes and degrading proteoglycan, is preferred. Examples of the above-mentioned retinoid having an inhibitory activity on inflammatory cytokine production and an inhibitory activity on MMP-3 production include retinoic acid, retinol, retinol palmitate and the like.

In addition, methods for confirming a retinoid's inhibitory activities on inflammatory cytokine production and MMP production can be performed with reference to Example 4 described later.

In order to transport a retinoid serving as the abovementioned medicinal ingredient to the articular synovial membrane/chondrocyte without irritating the skin, for the agents of the present invention, the retinoid is encapsulated in an inorganic-salt nanocapsule. In consideration of permeability to the skin, the particle size of the capsule is in nano scale, specifically 1 nm to 400 nm, preferably 5 nm to 300 nm, more preferably 5 nm to 200 nm, and yet more preferably 10 nm to 100 nm.

The structure of the capsule comprises a shell made of a polyvalent metal inorganic salt which envelopes a retinoid, and on the surface of the shell, a hydrophilic group for inhibiting interparticle aggregation.

The polyvalent metal inorganic salt mentioned above is preferably a divalent or trivalent metal inorganic salt, and more preferably a divalent metal inorganic salt. Examples of the divalent and trivalent metal include calcium (divalent), magnesium (divalent), zinc (divalent), iron (divalent and trivalent), copper (divalent and trivalent) and the like. These metal salts can also be used in an appropriate salt form, such as a carbonate salt, a phosphate salt, and a sulfate salt, according to the type of metal. Of them, calcium phosphate, calcium carbonate, magnesium carbonate, magnesium phosphate, zinc carbonate, zinc phosphate, iron sulfate, copper sulfate, and the like can be suitably used. Of them, calcium carbonate, zinc carbonate, and calcium phosphate (apatite) are more suitable when biocompatibility is considered.

As described later, the capsule of a polyvalent metal salt constituting the shell is prepared using as raw materials, a solution containing polyvalent metal ions and a salt that is dissociable into polyvalent inorganic anions.

The hydrophilic group is not specifically limited so long as it can be projected or exposed on the shell surface to inhibit interparticle aggregation, and examples thereof can include polyoxyethylene chains and sugar chains such as sucrose and dextran. In order to project or expose a hydrophilic group on the surface of the nanocapsule of the present invention, for example, before coating retinoid micelle particles with the above polyvalent metal salt, a nonionic surfactant having the above hydrophilic group may be added to a solution where the retinoid micelle particles are dispersed. In a case where the hydrophilic group of the present invention is a polyoxyethylene chain (—O—(C₂H₄O)ₙH)), a nonionic surfactant whose polymerization degree (n) is 4 to 100, or 10 to 100, and preferably 20 to 80, or 30 to 80, is used.

The method for preparing the above retinoid-containing nanocapsule can be carried out as follows.

First, a retinoid is dissolved in a small amount of a highly polar organic solvent in the presence of a nonionic surfactant, and then dispersed in water containing a strong alkali such as sodium hydroxide to form spherical micelles of the retinoid in water.

Examples of the organic solvent that can be used herein may include ethanol, methanol, acetone, ethyl acetate, dimethyl sulfoxide and the like. Of them, ethanol and methanol can be suitably used in producing the agents of the present invention, since they are highly soluble and less irritating to the skin.

The nonionic surfactant should have a hydrophilic group which can prevent the micelle from insolubilization when polyvalent metal ions are added at a subsequent process, as well as the aggregation of particles and precipitation of aggregated particles that ensue. For example, when a polyoxyethylene group is provided on the surface of the capsule as a hydrophilic group, for example, a polyoxyethylene-based nonionic surfactant may be used as a nonionic surfactant. Examples of a polyoxyethylene-based nonionic surfactant may include, for example, polyoxyethylene (20) sorbitan monooleate (Tween 80), polyoxyethylene (20) sorbitan monostearate (Tween 60), polyoxyethylene (20) sorbitan monopalmitate (Tween 40), polyoxyethylene (20) sorbitan trioleate (Tween 85), polyoxyethylene (8) octylphenylether, polyoxyethylene (20) cholesterol ester, polyoxyethylene hydrogenated castor oil and the like. That is, in the case of Tween 80, mixed micelles are formed with a retinoid, and highly hydrophilic polyoxyethylene chains are projected on the micelle surface. This projection of polyoxyethylene chains prevents the aggregation of micelles when the micelles are covered with polyvalent metal ions at a subsequent process.

Moreover, for example, a sucrose fatty acid ester can be used as an example of another nonionic surfactant.

A polyvalent metal ion aqueous solution is added to the above reaction solution. Since the surface of the micellized retinoid is covered with negative charges, the polyvalent metal ions added therein are adhered onto the micelle surface.

Aqueous solutions of calcium chloride, magnesium chloride, zinc chloride, iron chloride, copper chloride and the like can be used as a polyvalent metal ion aqueous solution. As compared to strong alkali such as sodium, polyvalent ions such as calcium, magnesium, zinc, iron, and copper are more tightly absorbed (bound) to micelles, and replacement of sodium ions takes place. As a result, a large number of polyvalent ions are absorbed (bound) to the surface of the retinoid micelles to form micelles having surfaces in a spherical or oval shape, or the like.

In order to neutralize the charge on the micelle surface, a salt dissociable into polyvalent inorganic anions, such as a carbonate salt, a phosphate salt, and a sulfate salt is added to the solution containing micelles covered with polyvalent metal ions. Sodium carbonate as a carbonate salt, sodium phosphate as a phosphate salt, sodium sulfate as a sulfate salt and the like can be used. The addition of these carbonate salt and phosphate salt leads the complete neutralization of charge on the micelle surface and the absorption (binding) of carbonate ions, phosphate ions, or sulfate ions to the micelle surface, to thereby construct a crystal of a polyvalent metal salt, that is, a shell enclosing retinoid and precovered with the polyvalent metal ions.

The nanocapsule of the present invention is coated with a polyvalent metal inorganic salt, and is reasonably water-soluble. When calcium carbonate is prepared by a common precipitation method, it takes on a crystal form called calcite, which has extremely low water-solubility. However, when a polyvalent metal inorganic salt is formed on the surface of micelle particles by the abovementioned method, the inorganic salt hardly takes on a rigid crystal structure because of the spherical or oval shape of the micelle and instead, it has an amorphous structure or a metastable phase vaterite structure. When calcium carbonate is formed amorphous, it has high water solubility because it is not a rigid crystal structure, and can maintain satisfactory biodegradability and release retinoid as an active ingredient even when administered into a living body. Moreover, when calcium carbonate is formed as a vaterite, it can be readily degraded in the living body and can release retinoid because it has a higher water solubility is higher than other crystal structures calcite and aragonite.

The molar ratio of the first added polyvalent metal salt to the later added salt which is dissociable into polyvalent inorganic anions is preferably 1:0.05 to 0.33, and more preferably 1:0.2. For example, take calcium chloride and sodium carbonate as examples for explanation. If the molar ratio of calcium chloride to sodium carbonate is set 1:1, the solution gradually becomes turbid after 30 minutes or more, forming a precipitate after about 3 hours. On the other hand, if the ratio of added calcium chloride to sodium carbonate is set 1:0.05 to 0.33, particularly 1:0.2 or less, the reaction solution is kept transparent, and no precipitation is observed even after a long period of agitation. Such turbidity and precipitation occur because the diameters of micelle particles are large. Since the large particle size lowers skin permeability, the composition ratio can be determined according to the presence/absence of turbidity and precipitation.

As described above, the particle diameter of the constituted capsule with encapsulated retinoid is as small as nano-size, making it permeable from the periarticular skin to the joint. Then, in the joint where the capsule has penetrated, the retinoid within the capsule acts to induce hyaluronic acid production and collagen production in the synovial membrane and chondrocyte. Moreover, since the retinoid is coated with a polyvalent metal inorganic salt, the retinoid is released in a sustained manner, and therefore a sustained effect can be expected. Accordingly, the capsule can be suitably used as a less-burdensome therapeutic agent for patients.

The dosage form of external preparations comprising the retinoid-containing capsule can be appropriately selected from an ointment, a cream, a patch, a poultice, and the like. In the case of an external preparation, the formulation can be performed by appropriately mixing other ingredients, additives and the like, according to known production methods. As an additive, ingredients commonly used for skin external preparations can be appropriately mixed, such as surfactants, humectants, lower alcohols, water, thickeners, oils, UV-absorbers, fragrances, antioxidants, chelating agents, dyes, preservatives, and antifungal agents.

Retinoid-containing capsules of the present invention can be used as therapeutic agents for local inflammation. When applied to the skin, the present invention's therapeutic agents for local inflammation penetrate into the joint and induce hyaluronic acid and collagen production in the joint. In addition, it was also shown that the therapeutic agents can lower the values of inflammatory cytokines (such as IL-6, IL-1α, and TNF-α) in the blood and lower MMP-3 in the blood by applying the agents to the skin. Accordingly, the present agents are also effective as therapeutic agents for inflammations caused by inflammatory cytokines, and accompanying diseases of the MMP-3-induced degradation of cartilage tissue and proteoglycan. Diseases that accompany high production of inflammatory cytokines and MMP-3 include rheumatoid arthritis and osteoarthritis. For example, when the agents of the present invention are used for rheumatoid arthritis, it is expected that inflammation is alleviated by inhibiting inflammatory cytokine production, destruction of chondrocyte is suppressed by inhibiting MMP-3 production, and further cartilage tissue regeneration is enhanced by inducing collagen production.

Moreover, retinoid-containing capsules of the present invention are also useful as preventive and/or therapeutic agents for joint diseases accompanied by a decrease in intra-articular hyaluronic acid level. Retinoid-containing capsules of the present invention have been proven to induce hyaluronic acid to thereby improve the articular functions, and thus can be used as articular function-improving agents. Examples of the joint diseases that accompany a decrease in intra-articular hyaluronic acid level may include scapulohumeral periarthritis and knee joint pain in chronic rheumatoid arthritis, as well as osteoarthritis.

All prior art documents cited in the present specification are incorporated herein by reference.

### EXAMPLES

Herein below, the present invention will be described with reference to Examples, but it is not construed as being limited to these Examples.

### Example 1

### Preparation of Retinoic Acid Nanocapsule-Blended External Preparations

Using raw materials shown in Table 1 below, nanocapsules comprising a retinoic acid (all-trans form: at) as an active ingredient (hereunder, referred to as “retinoic acid nanocapsules”) were prepared.

TABLE 1

| | Composition A | | Composition B | | Composition C | |
|---|---|---|---|---|---|---|
| All-trans retinoic | 140 | mg | 280 | mg | 560 | mg |
| acid (atRA) | | | | | | |
| Ethanol | 400 | μL | 800 | μL | 1600 | μL |
| 1N aqueous NaOH | 560 | μL | 1120 | μL | 2240 | μL |
| solution | | | | | | |
| Glycerin | 5 | mL | 5 | mL | 5 | mL |
| Distilled water | 17.72 | mL | 16.76 | mL | 14.28 | mL |
| Nonionic | 2 | mL | 2 | mL | 2 | mL |
| surfactant*¹⁾ | | | | | | |
| 5M aqueous MgCl₂ | 46.5 | μL | 93 | μL | 186 | μL |
| or CaCl₂ solution | | | | | | |
| 1M aqueous Na₂CO₃ | 46.5 | μL | 93 | μL | 186 | μL |
| solution | | | | | | |

Predetermined amounts of retinoic acid, ethanol and an aqueous NaOH solution were added into a reaction container, and were homogeneously dissolved. Glycerin and a nonionic surfactant (*1: Tween 80 was used) were added to this dissolved solution, and stirred for about 10 minutes. Then distilled water was further added thereto, and stirred for about 10 minutes. Next, an aqueous MgCl₂ or CaCl₂ solution was added to this solution, and stirred for about 1 hour. Furthermore, an aqueous Na₂CO₃ solution was added thereto, and stirred for about 1 hour.

By the above operation, retinoic acid nanocapsules having a magnesium carbonate or calcium carbonate coating formed around retinoic acid particles were obtained.

In order to prepare an ointment comprising the above retinoic acid nanocapsule, the reaction solution containing the above retinoic acid nanocapsule was freeze-dried for one day and night. A predetermined amount of white vaseline was added to the dried paste, and mixed and stirred to complete an external preparation (ointment) of the retinoic acid nanocapsule.

In addition, all processes of the nanocapsule preparation and ointment preparation mentioned above were performed under light shielding. Moreover, an injectable solution with retinoic acid nanocapsule suspended in a physiological salt solution was also prepared as an in vitro experimental formulation to be used for the following Examples.

### Example 2

### Study of the Induction of Hyaluronic Acid Production by In Vitro Cell Culture Experiment

Synovial cells from OA patients were cultured, and the retinoic acid nanocapsule containing calcium carbonate as an ingredient of the capsule produced in Example 1, and retinoic acid (atRA) were added thereto. Six hours after the addition, colloidal iron staining was performed to confirm the status of hyaluronic acid production. The evaluation groups were: DMSO as control, atRA, and the retinoic acid nanocapsule. The concentration of added retinoic acid was set 0.05% in both groups of atRA and retinoic acid nanocapsule.

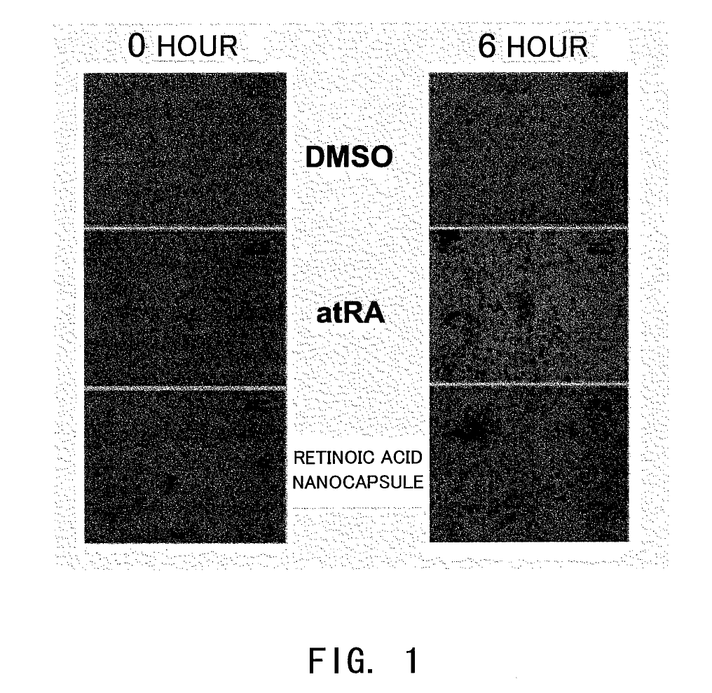

In FIG. 1, the black stains represent hyaluronic acid. Hyaluronic acid production was confirmed after 6 hours, in both groups added with atRA and the retinoic acid nanocapsule.

### Example 3

### Study of the Effect of Retinoic Acid Administration by In Vivo Experiment

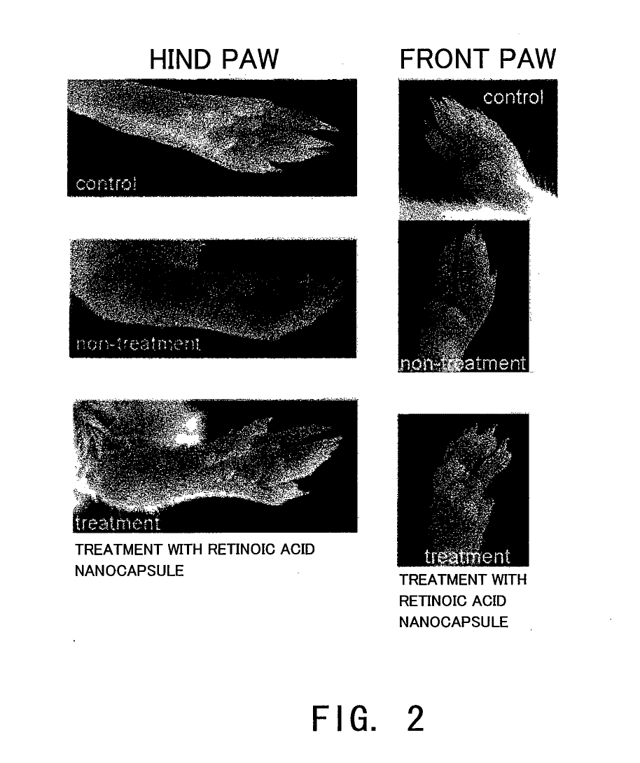

Arthritis model mice were produced according to a known method (Terato et al., J. Immunol. 148:2103-2108 (1992), Terato et al., Autoimmunity 22: 137-147 (1995)). Specifically, a monoclonal antibody cocktail (purchased from Immuno-Biological Laboratories Co., Ltd.) and LPS were used to elicit arthritis in BALB/c mice (seven-week-old, male). After the elicitation of arthritis, 30 mg of the external preparation of retinoic acid nanocapsules (containing 0.1% retinoic acid (atRA)) prepared in Example 1 was applied once daily to the front paw joint and hind paw joint of the mice for six days. Photographs of the paws taken at that time are shown in FIG. 2. The non-treatment group was observed to have apparent swellings on the fingers, while it was apparent that the treatment group was very close to the arthritis-free condition. The front paws showed similar results to the hind paws.

### Example 4

### Study of Blood Concentrations of MMP-3, IL-6, IL-1α, and TNF-α and Collagen Level in the Joints of Mice Administered with Retinoic Acid

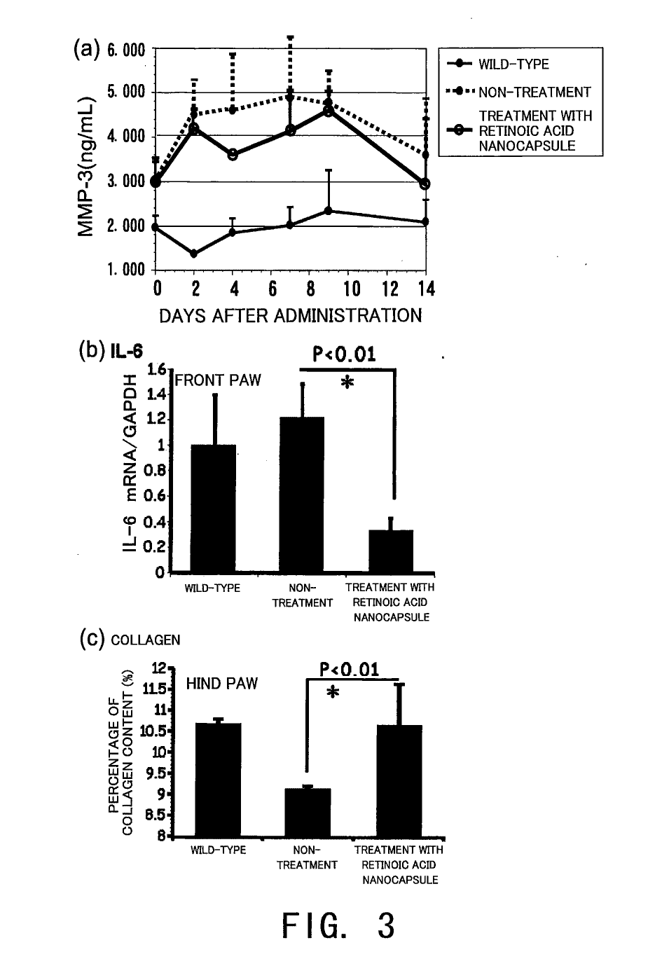

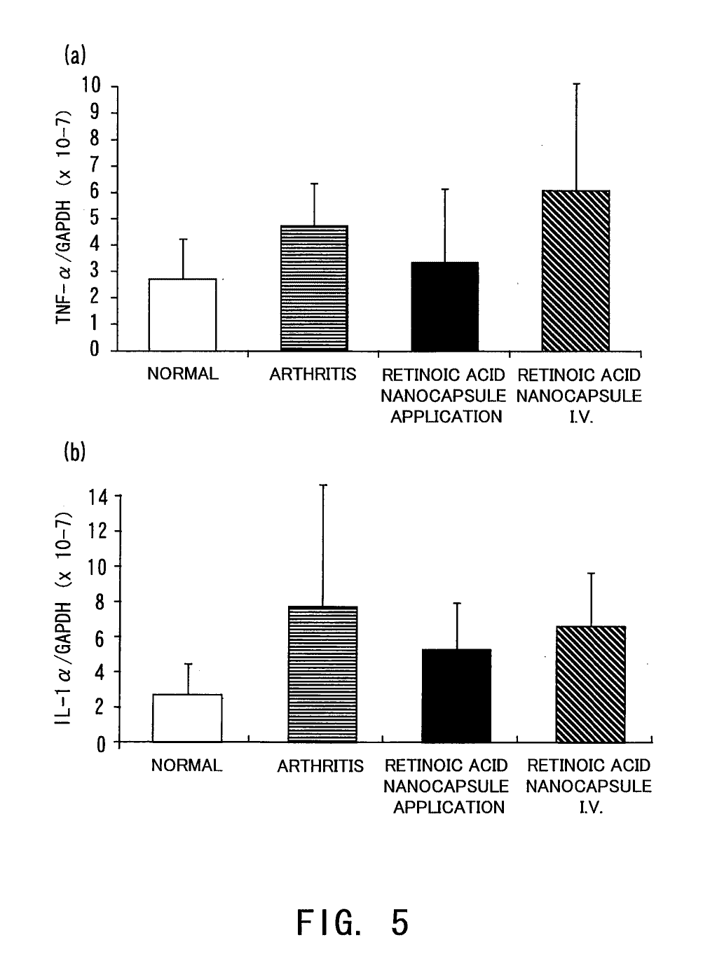

Similarly as in Example 3, arthritis was elicited in BALB/c mice (seven-week-old, male) using a monoclonal antibody cocktail and LPS. After the elicitation, 30 mg of the external preparation of retinoic acid nanocapsules (containing 0.1% retinoic acid (atRA)) prepared in Example 1 was applied once daily to the front paw joint and hind paw joint of the mice for six days. The respective blood concentrations of MMP-3, IL-6, IL-1α, and TNF-α and the collagen level in the joints were measured. The measured results of MMP-3, IL-6, and the collagen level are shown in FIG. 3, and the measured results of IL-1α and TNF-α are shown in FIG. 5.

In addition, MMP-3 was measured using an ELISA Kit for mouse MMP-3 measurement, “Quantikine (96 well)” (R&D SYSTEMS). The expression levels of IL-6, IL-1α, and TNF-α were obtained by measuring the mRNA levels by the real-time PCR method. The respective primer sequences are shown below.

GAPDH (internal standard gene as an index)

| | |
|---|---|
| Forward primer: | |
| 5′-TGAACGGGAAGCTCACTGG-3′ | (SEQ ID NO: 1) |
| Reverse primer: | |
| 5′-TCCACCACCCTGTTGCTGTA-3′ | (SEQ ID NO: 2) |
| IL-6 | |
| Forward primer: | |
| 5′-CCAGAGTCCTTCAGAGAGA-3′ | (SEQ ID NO: 3) |
| Reverse primer: | |
| 5′-GATGGTCTTGGTCCTTAGC-3′ | (SEQ ID NO: 4) |
| IL-1α | |
| Forward primer: | |
| 5′-ATGCAAGCTATGGCTCACTTCA-3′ | (SEQ ID NO: 5) |
| Reverse primer: | |
| 5′-GCTGATCTGGGTTGGATGGT-3′ | (SEQ ID NO: 6) |
| TNF-α | |
| Forward primer: | |
| 5′-ACGTGGAACTGGCAGAAGAG-3′ | (SEQ ID NO: 7) |
| Reverse primer: | |
| 5′-CTCCTCCACTTGGTGGTTTG-3′ | (SEQ ID NO: 8) |

Moreover, the collagen level was measured by conversion from the hydroxyproline level according to a known method (NAGATANI Yasunori, MUTO Yoshiaki, SATO Hiroshi, and IIJIMA Masao, An Improved Method for the Determination of Hydroxyproline, Journal of the Pharmaceutical Society of Japan, Vol. 106, pp. 41-46 (1986)).

As shown in FIG. 3, immediately after administration of the external preparation of retinoic acid nanocapsule, MMP-3 showed constantly lower values as compared with the non-treatment group, and the low values were more apparent after 14 days (FIG. 3(a)). The collagen level in the group administered with the external preparation of retinoic acid nanocapsule also increased to a degree close to normal value (FIG. 3(c)). This can be considered to reflect the fact that the MMP-3 value was constantly low.

Moreover, it was shown that application of the external preparation of retinoic acid nanocapsules also remarkably inhibited IL-6 in the blood (FIG. 3(b)). Furthermore, the values of TNF-α and IL-1α were also shown to be lowered by application of the external preparation of retinoic acid nanocapsule, as compared with the non-treated arthritis mice. In addition, experiments for a group intravenously injected with the retinoic acid nanocapsule were also conducted in parallel. In the intravenous administration group (i.v.), no inhibitory effect on the production of IL-1α and TNF-α was observed, showing that the local administration by means of an external preparation is effective for inhibiting the production of TNF-α and IL-1α.

The above results, which showed inhibition of the production of MMP-3, IL-6, TNF-α, and IL-1α, suggest that the external preparation of retinoic acid nanocapsule is effective not only for inflammatory arthritis but also for rheumatoid arthritis.

### Example 5

### Situation of Hyaluronic Acid Production in the Joints of Mice Administered with Retinoic Acid

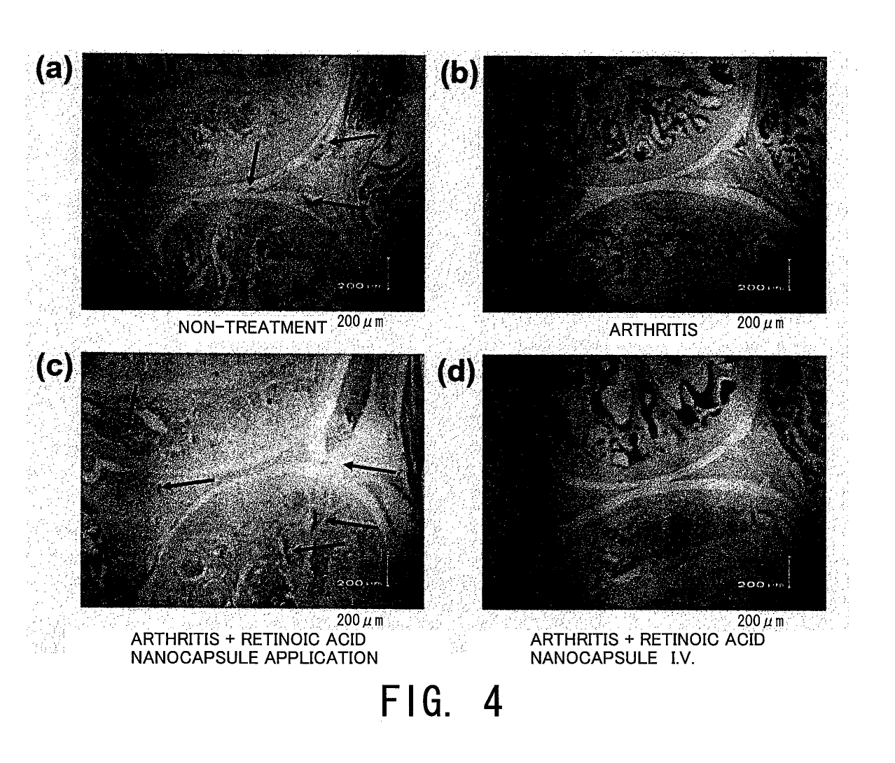

Similarly as in Example 3, arthritis was elicited in BALB/c mice (seven-week-old, male) using a monoclonal antibody cocktail and LPS. After the elicitation, 30 mg of the external preparation of retinoic acid nanocapsules (containing 0.1% retinoic acid) was applied once daily to the front paw joint and hind paw joint of the mice for 14 days. FIG. 4 shows the results of colloidal iron staining of the histological sections of the hind paw joints at that time.

In a normal mouse, it is apparent that hyaluronic acid was produced in the cavities between cartilages (FIG. 4(a)). The specimen with elicited arthritis showed almost no hyaluronic acid production (FIG. 4(b)). In the specimen applied with the external preparation of retinoic acid nanocapsule, the presence of hyaluronic acid was confirmed in the cartilage, and it was also elucidated that hyaluronic acid was produced and retained between chondrocytes (FIG. 4(c)) (the points of arrows indicate hyaluronic acid). Almost no such effect was exerted by the intravenous injection of the retinoic acid nanocapsule (FIG. 4(d)), proving that the retinoic acid nanocapsule is effective as an external preparation (local administration).

### INDUSTRIAL APPLICABILITY

The agents of the present invention can penetrate from periarticular skin to thereby induce intra-articular hyaluronic acid production. Furthermore, the agents can also inhibit the production of inflammatory cytokines that are inflammation-promoting factors, such as TNF, IL-1α, and IL-6, and inhibit the production of MMP-3 which has a degrading effect on articular cartilage and proteoglycan. Accordingly, the agents of the present invention enable one to conduct hyaluronic acid supplementation therapy, without performing repetitive injection into the joint or the like, which is a great burden in conventional therapeutic methods.

Moreover, since the agents of the present invention can also directly inhibit inflammatory cytokine production, a higher alleviating effect on inflammation can be expected as compared with conventional therapy of direct hyaluronic acid injection. Furthermore, since the agents inhibit production of MMP-3 which has an effect of destructing articular cartilage, an inhibitory effect on disease progression can also be expected as compared with conventional therapy of direct hyaluronic acid injection. The agents of the present invention with such inhibitory activity on inflammatory cytokine production and inhibitory activity on MMP production can be used as therapeutic agents for accompanying diseases of local inflammation such as rheumatoid arthritis, and as therapeutic agents for osteoarthritis and the like.

1. A therapeutic agent for local inflammation comprising an inorganic salt capsule which encapsulates a retinoid.

2. The therapeutic agent for local inflammation according to claim 1, wherein the inorganic salt capsule has a hydrophilic group and a polyvalent metal salt on its surface.

3. The therapeutic agent for local inflammation according to claim 1, wherein the retinoid is a retinoic acid, a retinol, or a retinol derivative.

4. The therapeutic agent for local inflammation according to claim 1, wherein the inorganic salt capsule is nano size.

5. The therapeutic agent for local inflammation according to claim 2, wherein the hydrophilic group is a polyoxyethylene group or a sugar chain.

6. The therapeutic agent for local inflammation according to claim 2, wherein the polyvalent metal is a divalent or trivalent metal.

7. The therapeutic agent for local inflammation according to claim 2, wherein the polyvalent metal is calcium, magnesium, zinc, aluminum, or copper.

8. The therapeutic agent for local inflammation according to claim 2, wherein the polyvalent metal salt is a carbonate, phosphate, or sulfate of the polyvalent metal.

9. The therapeutic agent for local inflammation according to claim 1, wherein the local inflammation is associated with the production of MMP and/or an inflammatory cytokine.

10. The therapeutic agent for local inflammation according to claim 9, wherein the MMP is MMP-3.

11. The therapeutic agent for local inflammation according to claim 9, wherein the inflammatory cytokine is TNF-α, IL-6, and/or IL-1α.

12. The therapeutic agent for local inflammation according to claim 1, wherein the local inflammation is arthritis.

13. The therapeutic agent for local inflammation according to claim 1, the agent is an external preparation.

14. A preventive and/or therapeutic agent for a joint disease that accompanies a decrease in intra-articular hyaluronic acid level, comprising as an active ingredient, a nanocapsule comprising a micelle particle that is formed by one or more retinoid-containing amphiphilic substances and coated with a polyvalent metal inorganic salt, wherein a hydrophilic group from at least one of the amphiphilic substances forming said micelle is at least partially present on the polyvalent metal inorganic salt-coated surface.

15. The preventive and/or therapeutic agent of claim 14, wherein one or more amphiphilic substances contains at least a retinoid and one or more nonionic surfactants.

16. The preventive and/or therapeutic agent of claim 15, wherein the nonionic surfactant is a polyoxyethylene-based nonionic surfactant or a sucrose fatty acid ester.

17. The preventive and/or therapeutic agent of claim 15, wherein the hydrophilic group of the nonionic surfactant is a polyoxyethylene chain.

18. The preventive and/or therapeutic agent of claim 14, wherein the joint disease that accompanies a decrease in intra-articular hyaluronic acid level is knee joint pain in osteoarthritis, scapulohumeral periarthritis, or chronic rheumatoid arthritis.

19. A method for preventing and/or or treating a joint disease or local inflammation that accompanies a decrease in hyaluronic acid level, comprising administration of the agent of claim 1.

20. (canceled)

21. The preventive and/or therapeutic agent of claim 15, wherein the joint disease that accompanies a decrease in intra-articular hyaluronic acid level is knee joint pain in osteoarthritis, scapulohumeral periarthritis, or chronic rheumatoid arthritis.

22. The preventive and/or therapeutic agent of claim 16, wherein the joint disease that accompanies a decrease in intra-articular hyaluronic acid level is knee joint pain in osteoarthritis, scapulohumeral periarthritis, or chronic rheumatoid arthritis.

23. The preventive and/or therapeutic agent of claim 17, wherein the joint disease that accompanies a decrease in intra-articular hyaluronic acid level is knee joint pain in osteoarthritis, scapulohumeral periarthritis, or chronic rheumatoid arthritis.

24. A method for preventing and/or treating a joint disease or local inflammation that accompanies a decrease in hyaluronic acid level, comprising administration of the agent of claim 14.


SEQUENCE LISTING (8 sequences)

SEQ ID NO: 1 - DNA, 19 bp, Artificial
  an artificially synthesized primer sequence
tgaacgggaagctcactgg

SEQ ID NO: 2 - DNA, 20 bp, Artificial
  an artificially synthesized primer sequence
tccaccaccctgttgctgta

SEQ ID NO: 3 - DNA, 19 bp, Artificial
  an artificially synthesized primer sequence
ccagagtccttcagagaga

SEQ ID NO: 4 - DNA, 19 bp, Artificial
  an artificially synthesized primer sequence
gatggtcttggtccttagc

SEQ ID NO: 5 - DNA, 22 bp, Artificial
  an artificially synthesized primer sequence
atgcaagctatggctcacttca

SEQ ID NO: 6 - DNA, 20 bp, Artificial
  an artificially synthesized primer sequence
gctgatctgggttggatggt

SEQ ID NO: 7 - DNA, 20 bp, Artificial
  an artificially synthesized primer sequence
acgtggaactggcagaagag

SEQ ID NO: 8 - DNA, 20 bp, Artificial
  an artificially synthesized primer sequence
ctcctccacttggtggtttg